(19) United States
(12) Patent Application Publication
(10) Pub. No.: US 2023/0302152 A1
(43) Pub. Date: Sep. 28, 2023

(54) CYCLODEXTRIN-BASED GAMMALINOLENIC ACID FORMULATION FOR TREATMENT OF BRAIN CANCER

(71) Applicant: SEFACOR INC., Somerville, MA (US)

(72) Inventor: DANIEL A. PEARSON, Bedford, NH (US)

(21) Appl. No.: 17/769,522

(22) PCT Filed: Oct. 9, 2020
(86) PCT No.: PCT/IB2020/059524

§ 371 Date: Apr. 15, 2022

Related U.S. Application Data
(60) Provisional application No. 62/915,841, filed on Oct. 16, 2019.

(51) Int. Cl. A61K 47/69; A61P 35/00

(52) U.S. Cl. CPC A61K 47/6907; A61P 35/00

(57) ABSTRACT

The present invention features a cyclodextrin-based gamma linolenic acid formulation for the treatment of brain cancer. More specifically, the invention relates to use of cyclodextrin analogs as carriers for gamma-linolenic acid and their pharmacological use. The present invention features GLA formulated as a cyclodextrin inclusion complex for the treatment of cancers of the brain, in particular glioblastoma multiforme and other gliomas, as well as metastatic cancers to the brain including leptomeningeal cancers. The inclusion complexes described herein take advantage of the ability of hydroxypropyl-beta-cyclodextrin (HPbCD), beta-cyclodextrin sulfobutyl ether (bCDSBE), and 2,6-dimethyl-beta-cyclodextrin (DMbCD) to complex with and subsequently solubilize GLA.

### FIELD OF THE INVENTION

The present invention features a cyclodextrin-based gamma linolenic acid formulation for the treatment of brain cancer. More specifically, the invention relates to use of cyclodextrin analogs as carriers for gamma-linolenic acid and their pharmacological use.

### BACKGROUND OF THE INVENTION

Each year, about 5-6 cases out of 100,000 people are diagnosed with primary malignant brain tumors, of which about 80% are malignant gliomas (MGs). Glioblastomamultiforme (GBM) accounts for more than half of MG cases. These cancers are associated with high morbidity and mortality. Despite current multimodality treatment efforts including maximal surgical resection if feasible, followed by a combination of radiotherapy and/or chemotherapy, the median survival is short - only about 15 months. Despite advances in treatment for newly diagnosed glioma patients, essentially all patients will experience disease recurrence. For patients with recurrent disease, conventional chemotherapy is generally ineffective with response rates <20%. There is also a high frequency of diffuse and leptomeningeal metastases from primary gliomas. With dismal prognoses and few effective treatments, new therapies are critically needed for brain cancer patients.

Several studies established the anti-tumor activity of gamma-linolenic acid (GLA) in gliomas [Naidu, et al., Prostaglandins, Leukotrienes, and Essential Fatty Acids (1992): Vol. 45(3), pages 181-184; Das, et al., Cancer Letters(1995): Vol. 94(2), pages 147-155; Reddy, et al., Journal of Clinical Neuroscience (1998): Vol. 5(1), pages 36-39; Bakshi, et al., Nutrition (2003): Vol. 19(4), pages 305-309; Das, Prostaglandins, Leukotrienes, and Essential Fatty Acids (2004): Vol. 70(6), pages 539-552; and Das, Medical Science Monitor (2007): Vol. 13(7), pages RA119-RA131] and this material has been extensively studied for its cytotoxic action on glioma cells both in vitro and in vivo [Farago, et al., Lipids in Health and Disease (2011): Vol. 10, page 173; Miyake, et al., Lipids in Health and Disease (2009): Vol. 8, page 8; and Benadiba, et al., IUBMB Life (2009): Vol. 61(3), pages 244-251]. These studies indicate that GLA has selective and specific tumoricidal action on glioma cells without harming the normal neuronal cells and low or no neurotoxicity both in animal tumor models and humans [Vartak, et al., British Journal of Cancer(1998): Vol. 77(10), pages 1612-1620;Leaver, et al., Prostaglandins, Leukotrienes, and Essential Fatty Acids (2002): Vol. 67(5), pages 283-292]. GLA also inhibits cancer cell invasion [Bell, et al., Journal of Neurosurgery(1999):Vol. 91(6), pages 989-996].

GLA not only has anti-cancer actions by itself, but also is also capable of potentiating the tumoricidal actions of other known anti-cancer drugs and radiation. [Vartak, et al., Lipids (1997): Vol.32(3), pages 283-292; Antal, et al., Lipids in Health and Disease (2014): Vol. 13, page 142 and Antal, et al., BiochimicaetBiophysicaActa (2015): Vol. 1851(9), pages 1271-1282]. GLA enhanced the cytotoxicity of the anti-cancer drugs, doxorubicin, cis-platinum and vincristine to HeLa cells in vitro [Sangeetha, et al., Medical Science Research (1993): Vol. 21, pages 457-459]. Drug uptake studies revealed that GLA gets incorporated into the cancer cell membranes to alter their fluidity and permeability and thus, enhance the anti-cancer drug uptake by HeLa cells. This may lead to an increase in the intracellular concentration of other anti-cancer drugs, thereby increasing their anti-cancer actions. In one example, GLA enhanced the cell growth inhibitory activity of vinorelbine on MCF-7 breast cancer cells in a dose-dependent manner [Menendez, et al., Breast Cancer Research and Treatment (2002): Vol. 72, pages 203-219]. In a similar fashion, GLA was also reported to increase the sensitivity of rat astrocytoma cells to radiation-induced cell kill [Vartak, et al., Lipids (1997): Vol. 32(3), pages 283-292]. This indicates that prior exposure to GLA may render tumor cells more sensitive to the cytotoxic actions of radiation and conventional anti-cancer drugs in patients with glioma.

One problem with the use of GLA as a therapeutic agent is that it is highly lipophilic and not easily formulated. Administering active agents that are not soluble in water poses a challenge that requires the use of an appropriate vehicle for bringing an effective amount of the active component to the desired place of action. Oil-in-water emulsions are commonly used for the delivery of active components that are not soluble in water. The emulsions that are conventionally used to deliver active components, however, suffer from a number of significant limitations and disadvantages. Emulsions are kinetically stable structures that are subject to destabilization through a number of mechanisms, ultimately resulting in complete phase separation of the emulsion. The tendency of emulsions to physically alter over time presents problems for their storage and handling. Furthermore this physical degradation increases the likelihood that the preparation is in a sub-optimal state when physically administered. What is needed is a stable formulation of GLA, where the formulated GLA is stable over time and readily administered to patients in need of treatment.

Cyclodextrins (CDs) are pharmaceutical excipients used in numerous pharmaceutical products worldwide. CDs form a subgroup of oligosaccharides consisting of several alpha(¼)-linked D-glucopyranose units. The arrangement of hydroxyl groups on the donut-shaped CD molecules bestows hydrophilic and hydrophobic domains on them, a polar exterior and a nonpolar interior. their central cavity enables encapsulation of nonpolar molecules or molecular moieties, a property that has been exploited in supramolecular analytical, food, and pharmaceutical chemistry. The most common natural CDs, and the only ones used in pharmaceutical products, are αCD, βCD and γCD consisting of 6, 7, and 8 D-glucopyranose units, respectively. In aqueous solutions CDs are able to form water-soluble inclusion complexes of lipophilic poorly-soluble drugs. In general, the complexation of fatty acids by various cyclodextrins is known, but the complexation of gamma-linolenic acid with the cyclodextrin analogs used to form the inclusion complexes of the present invention have yet to be described.

### SUMMARY OF THE INVENTION

Accordingly, the present invention features GLA formulated as a cyclodextrin inclusion complex for the treatment of cancers of the brain, in particular gliobastoma multiforme and other gliomas, as well as metastatic cancers to the brain including leptomeningeal cancers. The inclusion complexes described herein take advantage of the ability of hydroxypropyl-beta-cyclodextrin (HPbCD), beta-cyclodextrin sulfobutyl ether (bCDSBE), and 2,6-dimethyl-beta-cyclodextrin (DMbCD) to complex with and subsequently solubilize GLA.

In one aspect, the invention features a cyclodextrin (CD) inclusion complex of gamma-linolenic acid (GLA), where the concentration of GLA is in the range of 1-20 mg/ml and the concentration of CD is in the range of 10-40% (w/v).

In one embodiment, the cyclodextrin is selected from 2-hydroxypropyl-β-cyclodextrin (HPbCD); sulfobutyl ether-β-cyclodextrin, sodium salt (bCDSBE); and 2,6-dimethyl-β-cyclodextrin (DMbCD).

In another embodiment, the CD/GLA molar ratio is in the range of from about 2:1 to 8:1.

In a further embodiment, the CD/GLA molar ratio is about 4:1.

In one embodiment, the inclusion complex is a nanoemulsion. Nanoemulsions are colloidal particulate systems in which the particles range in diameter from 10 to 1,000 nanometers. In a further embodiment, the nanoemulsion particle size is in the range of about 50 nm to 300 nm.

In another embodiment, the CD/GLA complex is a lyophilizate.

In another aspect, the invention features a GLA/CD inclusion complex useful for the manufacture of a medicament for the treatment of cancer.

In one embodiment, the cancer is glioblastoma multiforme.

In another embodiment, the cyclodextrin of the medicament is selected from 2-hydroxypropyl-β-cyclodextrin; sulfobutyl ether-β-cyclodextrin, sodium salt; and 2,6-dimethyl-β-cyclodextrin.

In yet another embodiment, the medicament includes a CD/GLA inclusion complex where the molar ratio is in the range of from about 2:1 to 8:1 CD/GLA. In a further embodiment, the CD/GLA molar ratio is about 4:1.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a Transmission Electron Microscope (TEM) photograph of a 10 mg/mL solution of GLA in 20% HPbCD.

FIG. 2 shows the inhibition of U87 cell viability with a GLA/HPbCD inclusion complex.

FIG. 3 shows the inhibition of U87 cell viability with a GLA/bCDSBE inclusion complex.

FIG. 4 shows the inhibition of U87 cell viability with a GLA/DMbCD inclusion complex.

FIG. 5 shows a photograph of tumor vs. control in an orthotopic tumor model using a GLA/CD formulation of the invention.

FIG. 6 and FIG. 7 show photomicrographs of tumor vs. control in an orthotopic tumor model using a GLA/CD formulation of the invention.

### DETAILED DESCRIPTION OF THE INVENTION

Gamma-linolenic acid was obtained from the seeds of Borago officinalis that had been processed into borage oil, containing 22% GLA. This commercially available material was purified by methods known to those skilled in the art such that the resulting GLA purity was 98% or higher as analyzed by gas chromatography - flame ionization detection (GC-FID). Formulations used in the methods of the invention use GLA with this level of purity.

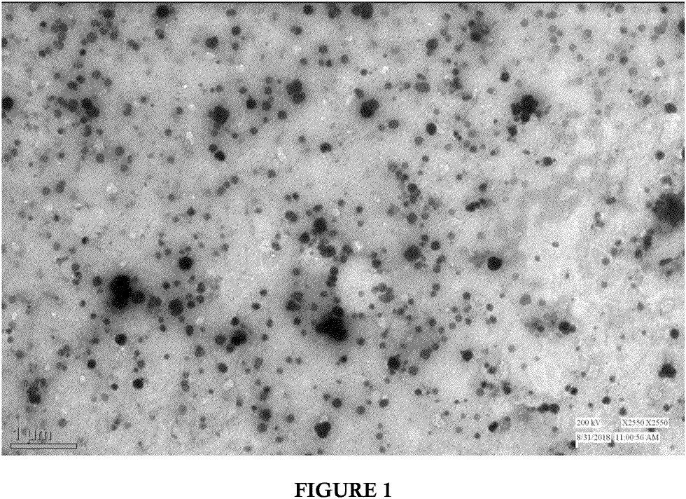

GLA was formulated as a cyclodextrin complex in either distilled water or in buffered solutions. Various cyclodextrins are useful for GLA formulation as indicated in the examples provided herein. Such cyclodextrins include 2,6-dimethyl-β-cyclodextrin (DMBCD), 2-hydroxypropyl-beta-cyclodextrin (HPbCD), or β-cyclodextrinsulfobutyl ether sodium salt (bCDSBE). In one example, 20 microliters of a 10 mg/mL GLA in 20% HPbCD solution was added to a carbon coated copper grid. The grid was air dried at room temperature and then viewed under Transmission Electron Microscope (Technai G2, Philips, Netherlands). The resulting photograph shows well dispersed droplets with average size of 200±85 nm. See FIG. 1.

Once mixed together to form a clear solution, water-soluble adduct can be lyophilized. Reconstitution of the lyophilizate yields as clear solution with the same properties as the formulation before lyophilization.

The following non-limiting examples are provided to further exemplify the invention.

### Example 1. Formulation of GLA/CD Inclusion Complexes

### (I) Preparation of 40% Cyclodextrin Solution

(a) 4.00 g of a cyclodextrin selected from 2,6-dimethyl-β-cyclodextrin (DMBCD), 2-hydroxypropyl-beta-cyclodextrin (HPbCD), or β-cyclodextrinsulfobutyl ether sodium salt (bCDSBE) was weighed into conical centrifuge tube. 0.01 M phosphate buffered saline (PBS, 5 mL at pH = 7.4) was added. The mixture was vortexed and/or sonicated until the solution became clear.

(b) Additional PBS was added to bring the volume in each tube up to 10.0 mL and the solution was mixed well.

(c) The solution was filtered through a 0.22 µm filter under sterile conditions and stored at ≤ 4° C.

### (ii) Preparation of Gamma-linolenic Acid (GLA) Suspension in H₂O

(a) 100 mg of GLA was placed into a 15 mL centrifuge tube.

(b) 4.9 mL of sterile water was added to the GLA and the tube was vortexed at high speed for 3 hours to produce a uniform suspension.

(c) Once the agitation of the suspension was complete, the GLA suspension was immediately apportioned into 2.0 mL Eppendorf tubes and mixed with various cyclodextrin analogs as indicated in Step (iii).

### (iii) Dilution and Agitation of GLA Test Samples

GLA/cyclodextrin dilutions were prepared under sterile conditions by adding the appropriate amount of the 40% CD solution, 20 mg/mL GLA suspension, PBS, and/or water to a 2 mL Eppendorf tube as indicated in Table 1. The mixtures were vortexed until a clear appearance was observed (usually about 15 seconds) before use.

TABLE 1

GLA/Cyclodextrin Dilutions

| | | | | |
|---|---|---|---|---|
| Tube No. (contents) | 40% CD Solution in PBS | 20 mg/mL GLA in H₂O | PBS | H₂O |
| 1 (10 mg/mL GLA/HPbCD, 0.005 M PBS) | 500 µL | 500 µL | -- | -- |
| 2 (HPbCD control, 0.005 M PBS) | 500 µL | -- | -- | 500 µL |
| 3 (10 mg/mL GLA/bCDSBE, 0.005 M PBS) | 500 µL | 500 µL | -- | -- |
| 4 (Bcdsbe control, 0.005 M PBS) | 500 µL | -- | -- | 500 µL |
| 5 (10 mg/mL GLA/DMbCD, 0.005 M PBS) | 500 µL | 500 µL | -- | -- |
| 6 (DMbCD control, 0.005 M PBS) | 500 µL | -- | -- | 500 µL |
| 7 (GLA control, 0.005 M PBS) | -- | 500 µL | 500 µL | -- |

### Example 2. U87 Cell Viability Assay

### (i) Preparation of 10 Mg GLA/mL in 20% Cyclodextrin Dilutions

Each of the cyclodextrin solutions from Table 1 were diluted in a 96 well plate with Eagle’s Minimum Essential Medium (EMEM) containing 10% fetal bovine serum (FBS) and 1% Pen/Strep as indicated in Table 2.

TABLE 2

Dilution of GLA/CD Solutions for U87 Cell Assay

| S. No. | Volume of medium (µL) | Test items or control | Dilutions | Concentration per dilution (µg/mL) (2X) |
|---|---|---|---|---|
| 1 | 1.86 mL | 144 µL | - | 720 |
| 2 | 150 | - | 150 µL from 1 | 360 |
| 3 | 150 | - | 150 µL from 2 | 180 |
| 4 | 150 | - | 150 µL from 3 | 90 |
| 5 | 150 | - | 150 µL from 4 | 45 |
| 6 | 150 | - | 150 µL from 5 | 22.5 |
| 7 | 150 | - | 150 µL from 6 | 11.3 |
| 8 | 150 | - | 150 µL from 7 | 5.6 |
| 9 | 150 | - | 150 µL from 8 | 2.8 |

### (ii) Testing in U87 Cell Assay

U87 cells in 100 µL of media were incubated individually in wells of a 96-well plate for 24 hours to ensure attachment. A 100 µL aliquot from each of the test article or cyclodextrin control dilutions was then added to the appropriate test well and the plates were incubated for 72 hours in a 5% CO₂ incubator at 37° C.

After the U87 cells were incubated in the presence of GLA/CD or control CD solutions, a CellTiter-Glo® Luminescent Cell Viability Assay from Promega was used to assess cell viability. Briefly, the CellTiter-Glo assay is a homogeneous method to determine the number of viable cells in culture based on quantitation of the ATP present, which is directly proportional to the number of metabolically active cells present in the culture medium. The assay relies on the properties of a thermostable luciferase, which generates a stable luminescent signal. The assay was performed as follows:
(1) The plate was removed from the incubator and equilibrated at room temperature for about 30 minutes;
(2) Cell media was carefully removed so as not to disturb the cells and replaced with 100 µL of media that did not contain fetal bovine serum;
(3) CellTiter-Glo® reagent (100 µL) was added to each well;
(4) The contents of the wells were mixed on an orbital shaker for 2 minutes to induce cell lysis;
(5) The plate was incubated at room temperature for 10 minutes; and
(6) The luminescence was recorded using a Perkin-Elmer EnVision multilabel reader.

### (iii) Effect of GLA/CD Solutions of Cells Viability

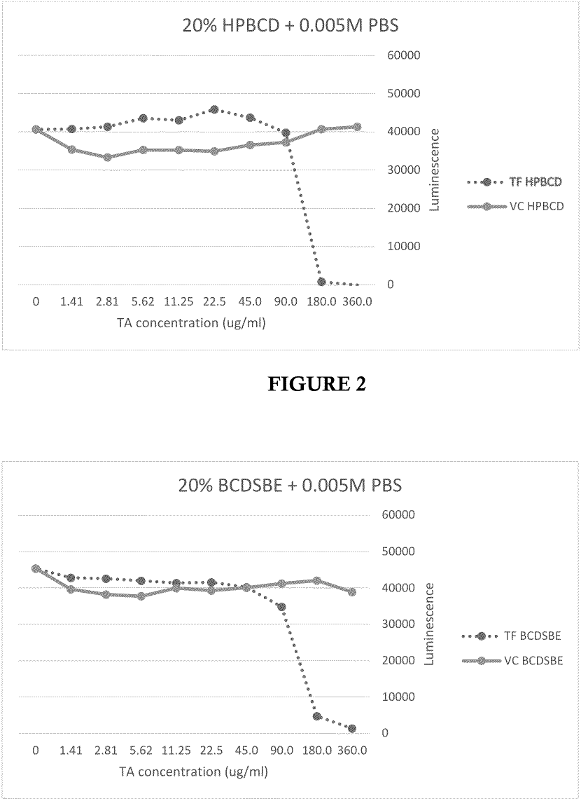

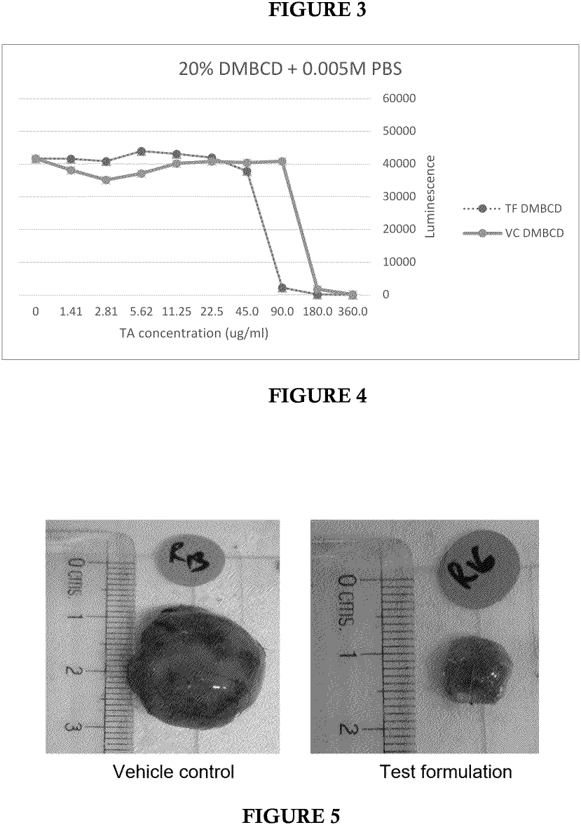

As shown in FIG. 2, where TF is the test article (GLA/20% HPbCD) and VC is the vehicle control (20% HPbCD alone), theIC₅₀of GLA/HPbCD= 117 µg/mL (420 µM). As shown in FIG. 3, where TF is the test article (GLA/20% bCDSBE) and VC is the vehicle control (20% bCDSBE alone), the IC₅₀ of GLA/bCDSBE= 117 µg/mL (420 µM). And as shown in FIG. 4, where TF is the test article (GLA/20% DMbCD) and VC is the vehicle control (20% DMbCD alone), the IC₅₀of GLA/DMbCD= 60 µg/mL (216 µM).

### Example 3. Preparation of GLA/HPbCD Inclusion Complexes in Artificial Cerebrospinal Fluid

Artificial cerebrospinal fluid (aCSF) and HPbCD emulsions in aCSF are prepared as follows.

### (i) Preparation of 20 mg/mL GLA in aCSF and in 20% HPbCD/aCSF

a) For 2x Solution A, the following amounts were dissolved in 250 mL sterile H₂O:NaCl (8.66 g); KCl (0.224 g); CaCl₂^{.}2H₂0 (0.206 g); and MgCl₂·6H₂O (0.163 g).

b) For 2x Solution B, the following amounts were dissolved in 250 mL sterile H₂O:Na₂HPO₄·7H₂O (0.214 g); andNaH₂PO₄·H₂O (0.027 g).

c) For 2x aCSF, Solutions A & B were combined in equal volumes.

### (ii) Preparation of 40% HPbCD in 2x aCSF

a) 2.00 g of hydroxypropyl-β-cyclodextrin (HPbCD) was weighed out in a sterile graduated 15 mL conical centrifuge tube; and 2.0 mL of 2x aCSF was added and the suspension was vortexed until the HPbCD has been totally wetted.

b) Additional 2x aCSF (about 1.85 mL) was added portion-wise to the HPbCD solution up to the 5.0 mL graduation mark, vortexing /sonicating after each addition until all of the HPbCD went into solution.

c) The solution was filtered through a 0.22 µm filter in a laminar flow hood.

### Example 4. Efficacy of a GLA/HPbCD Formulation in an Orthotopic Tumor Model

C6 glioma cells were grown in DMEM cell culture medium containing 10% fetal calf serum and antibiotics (penicillin 50 U/ml, streptomycin 50 ug/ml). Cells in the exponential phase of growth were used, growing in 75 cm² flasks in a humidified atmosphere of 5% CO₂/95% air at 37° C.

A subcutaneous injection was made on the lateral side of the back above the hind leg where 100 µL of Dulbecco’s phosphate-buffered saline (DPBS) containing 3 x 10⁶ C6 glioma cells were injected for the development of a flank tumor in Wistar rats. The animals were housed for 14-days during which the tumor size, weight of the animals were measured periodically. Following tumor development, the animals were randomly divided into 2 groups with 3 animals per group (n=3). A 100 µL intratumoral injections of a 10 mg/ml of GLA/HPbCD/aCSF formulation was administered on alternate days for 14 days to one group along with vehicle control to the other group. The weight of the animals, food/water intake, and tumor volume was noted periodically. At the end of the experiment, the rats were euthanized and the tumor was excised for histopathological analysis. The efficacy of the GLA/HPbCD/aCSF formulation was assessed by reduction of the tumor volume and histological evidence of regression of the tumor.

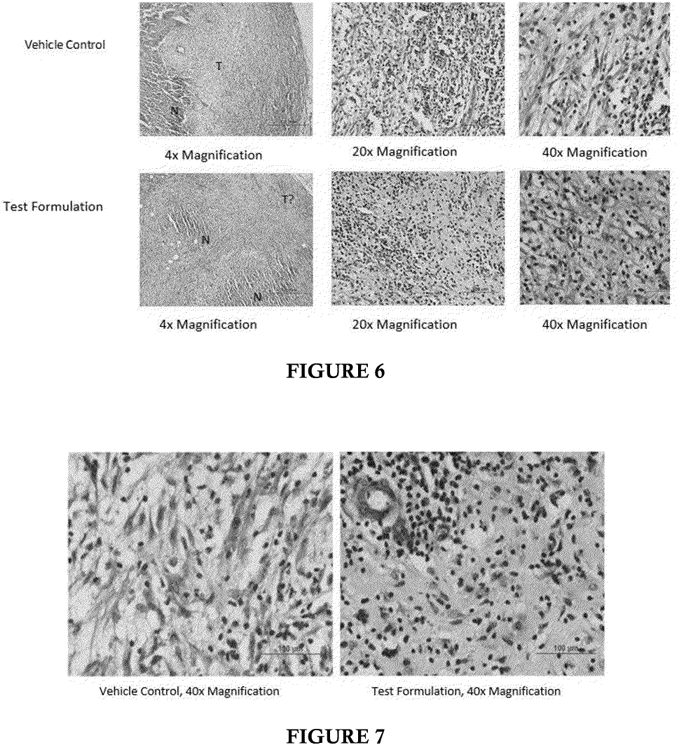

As shown in FIG. 5, a significant tumor regression (75 +/- 3.2%) was noted. The detailed histological examination of tumor of TF021 test formulation showed the area of tumor regression was infiltrated by foamy macrophages and lymphocytes, which was not seen in the vehicle control VC021 (FIG. 6).

We claim:

1. A cyclodextrin (CD) inclusion complex of gamma-linolenic acid (GLA), wherein
the concentration of GLA is in the range of 1-20 mg/ml;
the concentration of CD is in the range of 10-40% (w/v);
wherein the cyclodextrin is selected from 2-hydroxypropyl-β-cyclodextrin;
sulfobutylether-β-cyclodextrin, sodium salt; and 2,6-dimethyl-β-cyclodextrin; and
the inclusion complex is in the form of a nanoemulsion.

2. The inclusion complex according to claim 1, wherein the CD:GLA molar ratio is in the range of about 2:1 to 8:1.

3. The inclusion complex according to claim 3, wherein the CD:GLA molar ratio is about 4:1.

4. The inclusion complex according to claim 1, wherein the nanoemulsion particle size is in the range of about 50 nm to 300 nm.

5. The inclusion complex according to claim 1, wherein said complex is in the form of a lyophilizate.

6. Use of the inclusion complex according to claims 1 for the manufacture of a medicament for the treatment of cancer.

7. The medicament according to claim 6, wherein said cancer is glioblastoma multiforme.
